(19) United States
(12) Patent Application Publication
(10) Pub. No.: US 2011/0262980 A1
(43) Pub. Date: Oct. 27, 2011

(54) MICRO-ORGANISMS FOR THE PRODUCTION OF ACETOL OBTAINED BY A COMBINATION OF EVOLUTION AND RATIONAL DESIGN

(76) Inventors: Philippe Soucaille, Deyme (FR); Francois Voelker, Montrond Les Bains (FR); Rainer Figge, Riom (FR)

(21) Appl. No.: 12/532,531

(22) PCT Filed: Mar. 21, 2008
(86) PCT No.: PCT/EP08/53441

§ 371 Date: Sep. 22, 2009

(30) Foreign Application Priority Data
Mar. 23, 2007 (IB) PCTIB2007001682

(51) Int. Cl. C12P 7/26; C12N 1/15; C12N 1/19; C12N 1/21

(52) U.S. Cl. 435/148; 435/252.3; 435/254.11; 435/254.2; 435/252.33

(57) ABSTRACT

The present invention concerns a new method combining evolution and rational design for the preparation of a strain of micro-organism for the production of acetol from a simple carbon source. The said method comprises:
growing an initial strain under selection pressure in an appropriate growth medium, said initial bacterial strain comprising an attenuation of the expression of the tpiA gene and an attenuation the expression of at least one gene involved in the conversion of methylglyoxal to lactate, in order to promote evolution in said initial strain,
then selecting and isolating the evolved strain having an increased acetol or 1,2-propanediol production rate,
then reconstructing a functional tpiA gene in the evolved strain.

The present invention also concerns the evolved strain such as obtained, that may be furthermore genetically modified in order to optimize the conversion of a simple carbon source into acetol without by-products and with the best possible yield.

The present invention concerns a new method combining evolution and rational design for the preparation of a micro-organism to produce acetol, the micro-organism thereby obtained and its use for the preparation of acetol.

Acetol or hydroxyacetone (1-hydroxy-2-propanone) is a C3 keto alcohol, which is used as a reducing agent in vat dyeing process in the textile industry. It can advantageously replace traditional sulphur containing reducing agents in order to reduce the sulphur content in wastewater, harmful for the environment. Acetol is also a starting material for the chemical industry, used for example to make polyols or heterocyclic molecules. In addition, it possesses interesting chelating and solvent properties.

Currently, acetol is mainly produced by catalytic oxidation or dehydration of 1,2-propanediol. New processes starting from renewable feedstocks like glycerol have been proposed in DE 4128692 and WO 2005/095536. Currently, the production cost of acetol using chemical processes is too high to make a widespread industrial application feasible.

The disadvantages of the chemical processes for the production of acetol make biological synthesis an attractive alternative.

Acetol is the last intermediate in the synthesis pathway of 1,2-propanediol from sugars by microorganisms.1,2-propanediol is produced in the metabolism of common sugars (e.g. glucose or xylose) through the glycolysis pathway followed by the methylglyoxal pathway. Dihydroxyacetone phosphate is converted to methylglyoxal that can be reduced either to lactaldehyde or to acetol. These two compounds can then undergo a second reduction reaction yielding 1,2-propanediol. This route is used by natural producers of (R)-1,2-propanediol, such as Clostridium sphenoides and Thermoanaerobacter thermosaccharolyticum. Although the production of 1,2-propanediol has been investigated in these organisms, the production of acetol is not documented. Clostridium sphenoides is believed to produce 1,2-propanediol through lactaldehyde (Tran Din and Gottschalk, 1985). In Thermoanaerobacter thermosaccharolyticum, the intermediate in the production of 1,2-propanediol is acetol (Cameron and Cooney, 1986, Sanchez-Rivera et al, 1987). However, the genetic engineering in order to produce acetol with this last organism is likely to be limited due to the shortage of available genetic tools.

### PRIOR ART

The group of Cameron (Altaras and Cameron, 2000) and the group of Bennett (Bennett and San, 2001, Berrios-Rivera et al, 2003) have investigated the use of E. coli as a platform for metabolic engineering for the conversion of sugars to 1,2-propanediol. These studies rely on the one hand on the expression of one or several enzymatic activities in the pathway from dihydroxyacetone phosphate to 1,2-propanediol and on the other hand on the removal of NADH and carbon consuming pathways in the host strain. However, acetol was not investigated as a final product but only mentionned as one of the possible intermediates in the synthesis of 1,2-propanediol by the recombinant strains.

E. coli has the genetic capabilities to produce acetol. The biosynthetic pathway starts from the glycolysis intermediate dihydroxyacetone phosphate. This metabolic intermediate can be converted to methylglyoxal by methylglyoxal synthase encoded by mgsA gene (Cooper, 1984, Tötemeyer et al, 1998). Methylglyoxal is a C3 ketoaldehyde, bearing an aldehyde at C1 and a ketone at C2. Theses two positions can be reduced to alcohol by a methylglyoxal reductase activity, yielding respectively acetol and lactaldehyde (see FIG. 1). Misra et al (1996) described the purification in E. coli of two methylglyoxal reductase activities giving the same product acetol. One NADH dependent activity could be an alcohol dehydrogenase activity whereas the NADPH dependent activity could be a non-specific aldehyde reductase. Ko et al (2005) investigated systematically the 9 aldo-keto reducases of E. coli as candidates for the conversion of methylglyoxal into acetol. They showed that 4 purified enzymes, YafB, YqhE, YeaE and YghZ were able to convert methylglyoxal to acetol in the presence of NADPH. According to their studies, the methylglyoxal reductases YafB, YeaE and YghZ are the most relevant for the metabolism of methylglyoxal in vivo.

The production of acetol by genetically engineered yeast was reported in WO 99/28481. S. cerevisiae expressing the mgsA gene of E. coli was shown to produce acetol and 1,2-propanediol in flask culture. The best titers reported are below 100 mg/l acetol and 100 mg/l 1,2-propanediol. The two products are produced simultaneously.

An alternative method to obtain a strain producing acetol is to direct the evolution of an “initial strain” towards a state where the “evolved strain” produces the desired compound with better characteristics. This method is based on the natural evolution of a microorganism which is first modified by attenuation of two genes, tpiA and one gene involved in the conversion of methylglyoxal into lactate. The purpose for attenuating the tpiA gene coding for triose phosphate isomerase is to separate the two metabolic branches starting at glyceraldehyde-3-phosphate (GA3P) and dihydroxyacetone phosphate (DHAP) that are normally interconverted by this enzyme. The pathway from DHAP to acetol and 1,2-propanediol will be the “reducing branch” consuming reduced co-factors (NADH), whereas the metabolism from GA3P to acetate will be the “oxidative branch” producing NADH and energy for the growth of the cell. Without a functional tpiA gene, the metabolism of the cell is “locked” and the growth of the strain, the production of acetol and 1,2-propanediol and the production of acetate are tightly coupled. Under selection pressure in an appropriate growth medium, this initial strain will evolve to a state where the production of acetol and 1,2-propanediol by said strain is improved. This procedure to obtain an “evolved strain” of micro-organism for the production of acetol and 1,2-propanediol is described in the patent application WO 2005/073364. This technology is a clear improvement over the prior art. Further improvements for the production of acetol can be subsequently introduced in the evolved strain and specifically the suppression of the 1,2-propanediol production in order to accumulate only acetol.

The object of the present invention is the obtention of an acetol producer strain by evolution and subsequent rational genetic engineering of the evolved strain. A special feature is the reconstruction of a functional tpiA gene in the evolved tpiA minus strain. These modifications lead to an improved production of acetol.

### DESCRIPTION OF THE INVENTION

The present invention concerns a new method combining evolution and rational design for the preparation of a strain of micro-organism for the production of acetol from a simple carbon source. The said method comprises:
growing an initial strain under selection pressure in an appropriate growth medium, said initial bacterial strain comprising an attenuation of the expression of the tpiA gene and an attenuation the expression of at least one gene involved in the conversion of methylglyoxal to lactate (gloA, aldA and aldB), in order to promote evolution in said initial strain,
then selecting and isolating the evolved strain having an increased acetol or 1,2 propanediol production rate (increased by at least 20%),
then reconstructing a functional tpiA gene in the evolved strain.

In one aspect of the invention, the synthesis of unwanted by-products is attenuated by deleting the genes coding for enzymes involved in synthesis of lactate from pyruvate (ldhA), formate (pflA, pflB), ethanol (adhE). In another aspect of the invention, the Entner-Doudoroff pathway is eliminated by deleting either the edd or eda gene or both.

The microorganism used for the preparation of acetol is selected among bacteria, yeasts and fungi, but is preferentially from the species Escherichia coli or Klebsiella pneumoniae.

The present invention also concerns the evolved strain such as obtained, that may be furthermore genetically modified in order to optimize the conversion of a simple carbon source into acetol. In one aspect of the invention, the glyceraldehyde 3 phosphate activity is reduced in order to redirect a part of the available glyceraldehyde 3 phosphate toward the synthesis of 1,2-propanediol and/or acetol. In another aspect of the invention, the efficiency of the sugar import is increased, either by using a sugar import independent of phosphoenolpyruvate (PEP) like the one encoded by galP, or by providing more PEP to the sugar-phosphotransferase system. This is obtained by eliminating the pathways consuming PEP like pyruvates kinases (encoded by the pykA and pykF genes) and/or by promoting the synthesis of PEP e.g. by overexpressing the ppsA gene coding for PEP synthase.

Additionally, in order to prevent the production of 1,2-propanediol, the gldA gene coding for the enzyme involved in the conversion of acetol into 1,2-propanediol is attenuated.

Advantageously, the synthesis of the by-product acetate is prevented by attenuating one or several of the genes ackA, pta, poxB.

This invention is also related to a method for the production of acetol at an optimal yield, under aerobic, microaerobic or anaerobic conditions, using said evolved and optionally genetically modified strain. The produced acetol according to this method is subsequently recovered and optionally purified.

### BRIEF DESCRIPTION OF THE DRAWINGS

The accompanying drawing that is incorporated in and constitutes a part of this specification exemplifies the invention and together with the description, serves to explain the principles of this invention.

FIG. 1 depicts the genetic engineering of central metabolism in the development of an acetol production system from carbohydrates.

### DETAILED DESCRIPTION OF THE INVENTION

As used herein the following terms may be used for interpretation of the claims and specification.

The term ‘strain’ denotes a genetic variant of a species. Thus the term ‘strain of microorganism’ denotes a genetic variant of a species of a specific microorganism. The characteritics given for any strain apply also for the corresponding microorganism or vice versa.

According to the invention the terms ‘culture’, ‘growth’ and ‘fermentation’ are used interchangeably to denote the growth of bacteria in an appropriate growth medium containing a simple carbon source.

The term ‘simple carbon source’ according to the present invention denotes any source of carbon that can be used by those skilled in the art to support the normal growth of a micro-organism, and which can be hexoses, pentoses, monosaccharides, disaccharides, glycerol and combinations thereof. Preferentially, a simple carbon source can be : arabinose, fructose, galactose, glucose, lactose, maltose sucrose or xylose. A preferred simple carbon source is glucose

The term ‘appropriate growth medium’ according to the invention denotes a medium of known molecular composition adapted to the growth of the micro-organism and designed in such a way that it promotes the wanted evolution.

The evolution process according to the invention is a process for the preparation of evolved micro-organisms presenting improved production characteristics, and comprises the following steps:
a) Modification of a micro-organism to obtain an initial strain with a “locked” metabolism where the evolution can only take the desired direction when the cells of the initial strain are grown on an appropriate medium,
b) Growth of the initial strain obtained above on said appropriate medium in order to cause it to evolve, wherein the initial strain is grown under aerobic, micro-aerobic or anaerobic conditions,
c) Selection of the “evolved strains” able to grow under these specific conditions, presenting improved production characteristics for the desired compound.

This evolution process has been extensively described in the patent applications WO 2004/076659 filed on 17 Feb. 2004, and WO 2005/073364 filed on Dec. 1, 2005, by the same applicants.

The term ‘selection’ according to the invention denotes a process wherein the only strains of microorganisms that are retained in the culture medium are those presenting a better fitness under the selection pressure conditions. Typically, the fittest strains are outgrowing their competitors and are then selected. A simple way to select a specific evolved strain of microorganism in a population consists in growing the population in continuous culture in which slow-growing strains are eventually eluted from the culture. This is not an exclusive example for selection, and other methods of selection known by the expert in the field may be applied.

The term ‘isolation’ denotes a process where an individual strain presenting specific genetic modifications is separated from a population of strains presenting different genetic characteristics. This is done by sampling the biomass after the period of evolution and spreading it on Petri dishes to isolate single colonies.

The term “acetol or 1,2-propanediol production rate” means a production rate expressed in g/l/h, that is calculated as follows:

Concentration of acetol or 1,2-propanediol produced in the medium (g/l)/time necessary for this production (hour)

Additionally, a specific production rate expressed in g/g/h, taking into account the quantity of biomass can be calculated as follows:

Concentration of acetol or 1,2-propanediol produced in the medium (g/l)/concentration of biomass produced in the medium (g/l)/time necessary for these productions (h)

The concentration of biomass is determined either by measuring the absorbance of the fermentation broth with a spectrophotometer reading for example at 600 nm or by determining the dry weight of cells after drying a defined volume of fermentation broth.

The quantity of acetol or 1,2-propanediol produced is measured by high performance liquid chromatography (HPLC) with an adapted column according to a state of the art protocol.

In the present invention, evolved strains are selected for the following characteristics : an increased glucose uptake rate and an improved acetol or 1,2 propanediol production rate. The strains showing these characteristics are then isolated, and advantageously compared to each other, in the way to identify the best producer.

The glucose uptake rate, expressed in g/l/h is calculated as follow:

Concentration of glucose consumed by the culture (g/l)/time necessary for this consumption (h)

A specific glucose uptake rate can be calculated by taking into account the concentration of biomass in the medium, as previously described.

Glucose uptake rate and acetol or 1,2 propanediol production rate are intimately linked. If the consumption of glucose is increased, the production of the products from the glucose metabolism is increased in the same proportion.

After selection and isolation, the best evolved strains present a glucose uptake that is about 20% higher than the uptake of the initial strain, preferentially about 30% higher or more, more preferentially 50% higher.

The increased acetol or 1,2 propanediol production rate is of about 20% higher than the production rate of the initial strain, preferentially about 30% higher or more, more preferentially about 50% higher.

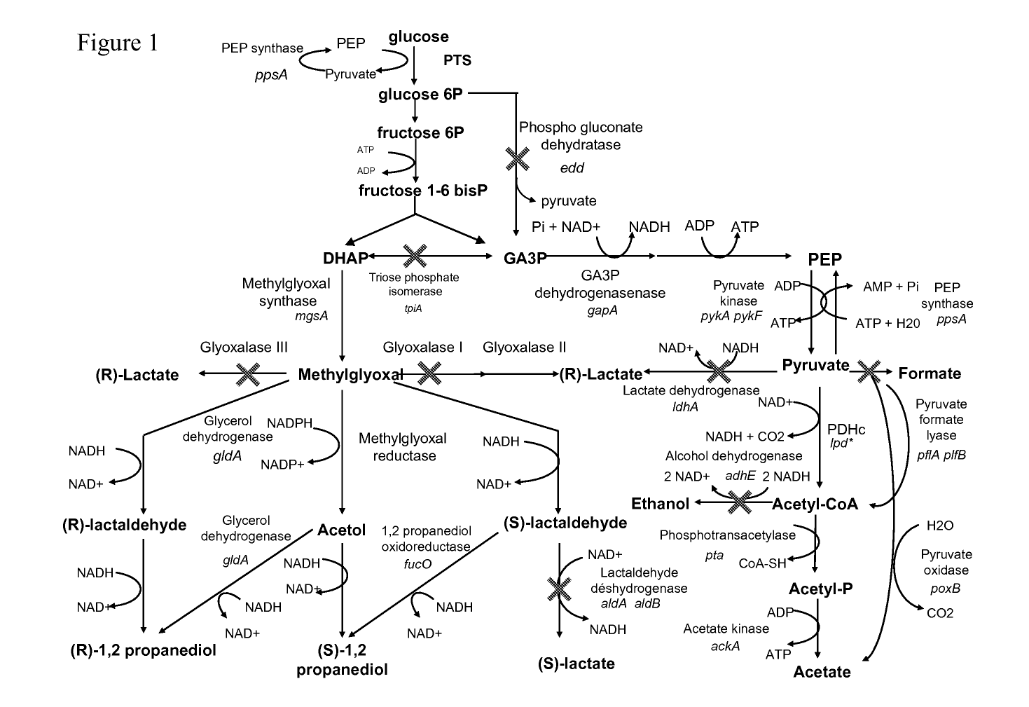

The tpiA gene encodes the enzyme ‘triose phosphate isomerase’, which catalyses the interconversion of DHAP and GA3P (see FIG. 1). The purpose of the attenuation of this gene is to engineer the metabolism of the cell in such a way that the evolution toward the most efficient acetol production becomes possible.

The term ‘attenuation of the expression of a gene’ according to the invention denotes the partial or complete suppression of the expression of a gene, which is then said to be ‘attenuated’. This suppression of expression can be either an inhibition of the expression of the gene, the suppression of an activating mechanism of the gene, a deletion of all or part of the promoter region necessary for the gene expression, or a deletion in the coding region of the gene. Preferentially, the attenuation of a gene is essentially the complete deletion of that gene, which gene can be replaced by a selection marker gene that facilitates the identification, isolation and purification of the strains according to the invention. A gene is preferentially inactivated by the technique of homologous recombination as described in Datsenko, K. A. & Wanner, B. L. (2000) “One-step inactivation of chromosomal genes in Escherichia coli K-12 using PCR products”. Proc. Natl. Acad. Sci. USA 97: 6640-6645. Other methods are described below.

The term “expression” refers to the transcription and translation of a gene sequence leading to the generation of the corresponding protein, product of the gene.

The term “reconstructing a functional tpiA gene in the evolved strain” means that the selected evolved strain is modified after the process of evolution by introducing a functional tpiA gene; this can be accomplished by replacing via homologous recombination the attenuated copy of the gene by a wild-type functional copy, thus restoring a triose phosphate isomerase activity similar to the activity measured in the initial strain, or by the introduction of a functional tpiA gene on a different chromosomal locus or by introducing a functional tpiA gene on a plasmid. This restoration can allow a yield of acetol production from glucose greater than 1 mole/mole by partly recycling GA3P into DHAP for the production of acetol through the action of triose phosphate isomerase.

The purpose of the attenuation of the expression of at least one gene involved in the conversion of methylglyoxal (2-oxo propanal) into lactate is to inhibit the conversion of methylglyoxal into lactate, so that the methylglyoxal present is used by the cell machinery essentially for the synthesis of acetol.

Genes involved in the conversion of methylglyoxal into lactate are in particular:
the gloA gene coding for glyoxalase I, catalysing the synthesis of lactoyl glutathione from methylglyoxal
the aldA and aldB genes coding for a lactaldehyde dehydrogenase (catalysing the synthesis of (S) lactate from (S) lactaldehyde).
   The expression of one or more of these genes is advantageously attenuated (or the gene is completely deleted) in the initial strain. Preferentially the gloA gene is deleted.

An additional modification is advantageously made to the initial strain consisting in suppressing the natural glucose fermentation routes, which consume the carbon source as by-products and therefore will lower the acetol yield.

In particular, it is advantageous to attenuate the expression of the gene ldhA coding for lactate dehydrogenase catalysing the synthesis of lactate from pyruvate, and the expression of the gene adhE coding for alcohol-aldehyde dehydrogenase catalysing the synthesis of ethanol from acetyl-CoA.

Similarly, it is possible to force the micro-organism to use the pyruvate dehydrogenase complex to produce acetyl-CoA and NADH from pyruvate. This can be achieved by attenuating the expression of genes pflA and pflB coding for pyruvate formate lyase.

Attenuation of at least one of the genes edd and eda coding for the enzymes involved in the Entner-Doudoroff pathway, is also useful to prevent the direct metabolism of glucose into glyceraldehyde-3-phosphate and pyruvate that can bypass the acetol synthesis pathway.

Preferentially, the initial strain is selected from the group consisting of bacteria, yeasts and fungi.

More preferentially, the initial strain is selected from the group consisting of Enterobacteriaceae, Bacillaceae, Streptomycetaceae and Corynebacteriaceae.

In a preferred embodiment of the invention, the initial strain is either Escherichia coli or Klebsiella pneumoniae.

The evolved strain susceptible to be obtained, and the evolved strain such as obtained by the process previously described, is also an object of the invention.

In this evolved strain, it is advantageous to modify the expression of specific genes, i.e. increasing or attenuating gene expression. These modifications allow to improve the acetol production performance.

To obtain an overexpression of a gene of interest, the man skilled in the art knows different methods, for example:

Replacement of the endogenous promoter with a stronger promoter

Introduction into the microorganism of an expression vector carrying said gene of interest.

Introducing additional copies of the gene of interest into the chromosome

The man skilled in the art knows several techniques for introducing DNA into a bacterial strain. A preferred technique is electroporation, which is well known to those skilled in the art.

In a specific embodiment of the invention, the evolved strain is modified by an attenuation of the glyceraldehyde 3 phosphate dehydrogenase (GAPDH) activity, in order to reduce the flux in the lower part of glycolysis and to redirect it toward the synthesis of DHAP and finally acetol (see FIG. 1). This decreased activity may in particular be obtained by an attenuation of the expression of the gapA gene.

The term “attenuation of the activity of an enzyme” refers to a decrease of activity of the enzyme of interest, compared to the observed activity in an evolved strain before any modification. The man skilled in the art knows numerous means to obtain this result, and for example:
Introduction of a mutation into the gene, decreasing the expression level of this gene, or the level of activity of the encoded protein.
Replacement of the natural promoter of the gene by a low strength promoter, resulting in a lower expression.
Use of elements destabilizing the corresponding messenger RNA or the protein.
Deletion of the gene if no expression at all is desired.

Advantageously in the evolved strain, the efficiency of sugar import is increased. A strong attenuation of the expression of the gapA gene resulting in a decrease of the carbon flux in the GAPDH reaction by more than 50%, this will result in the synthesis of less than 1 mole of phosphoenolpyruvate (PEP) per mole of imported glucose. The sugar-phosphotransferase system (PTS) usually assuring the import of simple sugars into the cell is coupled to a phosphorylation reaction giving glucose-6-phosphate. The phosphate needed for this reaction is provided by the conversion of PEP into pyruvate. Thus deacreasing the amount of PEP produced by reducing the flux through glyceraldehyde-3-phosphate reduces sugar import.

In a specific embodiment of the invention, the sugar might be imported into the microorganism by a sugar import system independent of phosphoenolpyruvate. The galactose-proton symporter encoded by the gene galP that does not involve phosphorylation can be utilized. In this case, the imported glucose has to be phosphorylated by glucose kinase encoded by the glk gene. To promote this pathway, the expression of at least one gene selected among galP and glk is increased. As a result the PTS becomes dispensable and may be eliminated by attenuating the expression of at least one gene selected among ptsH, ptsI or crr.

In another specific embodiment of the invention, the efficiency of the PTS is increased by increasing the availability of the metabolite PEP. Due to the attenuation of the gapA activity and of the lower carbon flux toward pyruvate, the amount of PEP in the modified strain of the invention could be limited, leading to a lower amount of glucose transported into the cell.

Various means exist that may be used to increase the availability of PEP in a strain of microorganism. In particular, a mean is to attenuate the reaction PEP→pyruvate. Preferentially, the expression of at least one gene selected among pykA and pykF, coding for the pyruvate kinases enzyme, is attenuated in said strain to obtain this result. Another way to increase the availability of PEP is to favour the reaction pyruvate→PEP, catalyzed by phosphoenolpyruvate synthase by increasing the activity of the enzyme. This enzyme is encoded by the ppsA gene. Therefore, preferentially in the microorganism the expression of the ppsA gene is increased. Both modifications can be present in the microorganism simultaneously.

In another specific embodiment of the invention, the synthesis of the by-product acetate is prevented. Under fully aerobic conditions, the reduced co-factor NADH is preferentially oxidised into NAD+ via the respiratory chain with oxygen as a terminal electron acceptor. Therefore, the synthesis of a co-product (e.g. acetate) is not mandatory. It is preferable to avoid such acetate synthesis to optimize the production of acetol.

To prevent the production of acetate, advantageously the activity of at least one enzyme involved in the synthesis of acetate is attenuated. Preferentially, the expression of at least one gene selected among ackA, pta and poxB is attenuated, all these genes coding for enzymes involved in different acetate biosynthesis pathways (see FIG. 1).

Preferentially in the evolved strain, the conversion of acetol into 1,2-propanediol is prevented by attenuating the activity of at least one enzyme involved in this conversion. More preferentially, the gldA gene, coding for glycerol dehydrogenase is attenuated.

Another object of the invention is a method for preparing acetol wherein an evolved strain such as described previously is grown in an appropriate growth medium containing a simple carbon source, and then the acetol produced is recovered. The production of acetol is performed under aerobic, microaerobic or anaerobic conditions.

The culture conditions (fermentation) for the micro-organisms according to the invention can be readily defined by those skilled in the art. In particular, bacteria are fermented at temperatures between 20° C. and 55° C., preferably between 25° C. and 40° C., and preferably at about 37° C. for E. coli and Klebsiella pneumoniae

This process can be carried out either in a batch process, in a fed-batch process or in a continuous process.

‘Under aerobic conditions’ means that oxygen is provided to the culture by dissolving the gas into the liquid phase. This could be obtained by (1) sparging oxygen containing gas (e.g. air) into the liquid phase or (2) shaking the vessel containing the culture medium in order to transfer the oxygen contained in the head space into the liquid phase. Advantages of the fermentation under aerobic conditions instead of anaerobic conditions is that the presence of oxygen as an electron acceptor improves the capacity of the strain to produce more energy in form of ATP for cellular processes. Therefore the strain has its general metabolism improved.

Micro-aerobic conditions are defined as culture conditions wherein low percentages of oxygen (e.g. using a mixture of gas containing between 0.1 and 10% of oxygen, completed to 100% with nitrogen), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The culture of strains, during the evolution process and the fermentation process for acetol production, is conducted in fermentors with a culture medium of known set composition adapted to the bacteria used, containing at least one simple carbon source. In particular, a mineral growth medium for E. coli or Klebsiella pneumoniae can thus be of identical or similar composition to M9 medium (Anderson, 1946, Proc. Natl. Acad. Sci. USA 32:120-128), M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as that defined by Schaefer et al. (1999, Anal. Biochem. 270: 88-96), and in particular the minimum culture medium named MPG described below:

| | | |
|---|---|---|
| K₂HPO₄ | 1.4 | g/l |
| Nitrilo Triacetic Acid | 0.2 | g/l |
| trace element solution* | 10 | ml/l |
| (NH₄)₂SO₄ | 1 | g/l |
| NaCl | 0.2 | g/l |
| NaHCO₃ | 0.2 | g/l |
| MgSO₄ | 0.2 | g/l |
| glucose | 20 to 100 | g/l |
| NaNO₃ | 0.424 | g/l |
| thiamine | 10 | mg/l |
| FeSO₄, 7H₂O | 50 | mg/l |
| yeast extract | 4 | g/l |

The pH of the medium is adjusted to 7.4 with sodium hydroxide.
*trace element solution: Citric acid 4.37 g/L, MnSO₄ 3 g/L, CaCl₂ 1 g/L, CoCl₂, 2H₂O 0.1 g/L, ZnSO₄, 7H₂O 0.10 g/L, CuSO₄, 5H₂O 10 mg/L, H₃BO₃ 10 mg/L, Na₂MoO₄ 8.31 mg/L.

Preferentially, the recovered acetol is furthermore purified. The man skilled in the art knows methods for recovering and purifying the produced acetol.

The invention is described above, below and in the Examples with respect to E. coli. Thus the genes that can be attenuated, deleted or over-expressed for the initial and evolved strains according to the invention are defined mainly using the denomination of the genes from E. coli. However, this designation has a more general meaning according to the invention, and covers the corresponding genes in other micro-organisms. Using the GenBank references of the genes from E. coli, those skilled in the art can determine equivalent genes in other organisms than E. coli.

The means of identification of the homologous sequences and their percentage homologies are well-known to those skilled in the art, and include in particular the BLAST programmes that can be used on the website http://www.ncbi.nlm.nih.gov/BLAST/ with the default parameters indicated on that website. The sequences obtained can be exploited (aligned) using for example the programmes CLUSTALW (http://www.ebi.ac.uk/clustalw/), with the default parameters indicated on these websites.

The PFAM database (protein families database of alignments and hidden Markov models http://www.sanger.ac.uk/Software/Pfam/) is a large collection of alignments of protein sequences. Each PFAM makes it possible to visualise multiple alignments, view protein domains, evaluate distributions among organisms, gain access to other databases and visualise known protein structures.

COGs (clusters of orthologous groups of proteins http://www.ncbi.nlm.nih.gov/COG/) are obtained by comparing protein sequences derived from 66 fully sequenced genomes representing 14 major phylogenetic lines. Each COG is defined from at least three lines, making it possible to identify ancient conserved domains.

### The Following Examples Show:

1—Construction of a modified strain of E. coli MG1655 lpd*, ΔtpiA, ΔpflAB, ΔadhE, ΔldhA, ΔgloA, ΔaldA, ΔaldB, Δedd, ΔarcA, Δndh
2—Evolution of said initial strain
3—Reconstruction of the tpiA gene in the selected evolved strain
4—Attenuation of the gapA gene; Deletion of the genes pykA, pykF and gldA; Overexpression of the ppsA gene
5—Deletion of the genes ackA-pta, poxB
6—Comparison of several obtained strains for acetol production under aerobic conditions
7—Production of acetol in fed-batch culture with the best strain.

### REFERENCES IN THE ORDER OF THE CITATION IN THE TEXT

1. Tran Din K and Gottschalk G (1985), Arch. Microbiol. 142: 87-92
2. Cameron DC and Cooney C L (1986), Bio/Technology, 4: 651-654
3. Sanchez-Rivera F, Cameron D C, Cooney C L (1987), Biotechnol. Lett. 9: 449-454
4. Altaras N E and Cameron D C (2000), Biotechnol. Prog. 16: 940-946
5. Bennett G N and San K Y (2001), Appl. Microbiol. Biotechnol. 55: 1-9
6. Cooper R A (1984), Annu. Rev. Microbiol. 38: 49-68
7. Tötemeyer S, Booth N A, Nichols W W, Dunbar B, Booth I R (1998), Mol. Microbiol. 27: 553-562
8. Misra K, Banerjee A B, Ray S, Ray M (1996), Mol. Cell. Biochem. 156: 117-124
9. Ko J, Kim I, Yoo S, Min B, Kim K, Park C (2005), J. Bacteriol. 187: 5782-5789
10. Berrios-Rivera S J, San K Y, Bennett G N (2003), J. Ind. Microbiol. Biotechnol. 30: 34-40
11. Datsenko K A and Wanner B L (2000), Proc. Natl. Acad. Sci. USA 97: 6640-6645
12. Anderson E H (1946), Proc. Natl. Acad. Sci. USA 32:120-128
13. Miller (1992), A Short Course in Bacterial Genetics: A Laboratory Manual and Handbook for Escherichia coli and Related Bacteria, Cold Spring Harbor Laboratory Press, Cold Spring Harbor, N.Y.
14. Schaefer U, Boos W, Takors R, Weuster-Botz D (1999), Anal. Biochem. 270: 88-96

### Examples

### Example 1

### Construction of a Modified Strain of E. coli MG1655 lpd*, ΔtpiA, ΔpflAB, ΔadhE, ΔldhA, ΔgloA, ΔaldA, ΔaldB, Δedd, ΔarcA, Δndh able to Evolve Toward Improved 1,2-propanediol Production

a) Construction of a Modified Strain E. coli MG1655 lpd*, ΔtpiA, ΔpflAB, ΔadhE, ldhA::Km, ΔgloA, ΔaldA, ΔaldB, Δedd

The chloramphenicol resistance cassette was eliminated in the strain E. coli MG1655 lpd*, ΔtpiA, ΔpflAB, ΔadhE, ldhA::Km, ΔgloA, ΔaldA, ΔaldB, Δedd::Cm (See WO2005073364) according to Protocol 1.

### Protocol 1 : Elimination of Resistance Cassettes

The chloramphenicol and/or kanamycin resistance cassettes were eliminated according to the following technique. The plasmid pCP20 carrying the FLP recombinase acting at the FRT sites of the chloramphenicol and/or kanamycin resistance cassettes was introduced into the strain by electroporation. After serial culture at 42° C., the loss of the antibiotic resistance cassettes was checked by PCR analysis with the oligonucleotides given in Table 1.

The presence of the modifications previously built in the strain was checked using the oligonucleotides given in Table 1.

The strain obtained was named E. coli MG1655 lpd*, ΔtpiA, ΔpflAB, ΔadhE, ldhA::Km, ΔgloA, ΔaldA, ΔaldB, Δedd.

TABLE 1
Oligonucleotides used for checking the insertion of a resistance
cassette or the loss of a resistance cassette

| | | | Homology with |
|---|---|---|---|
| Region name | Names of oligos | SEQ ID | chromosomal region |
| tpiA gene | cdh | N°1 | See WO2005073364 |
| (deletion) | YIIQ | N°2 | |
| pflAB gene | pflABF | N°3 | See WO2005073364 |
| | pflABR | N°4 | |
| adhE gene | ychGf | N°5 | See WO2005073364 |
| | adhECr | N°6 | |
| ldhA gene | hsIJC | N°7 | See WO2005073364 |
| (cassette insertion) | ldhAC2 | N°8 | |
| gloA gene | NemACd | N°9 | See WO2005073364 |
| | Rnt Cr | N°10 | |
| aldA gene | Ydc F C f | N°11 | See WO2005073364 |
| | gapCCr | N°12 | |
| aldB gene | aldB C f | N°13 | See WO2005073364 |
| | Yia Y Cr | N°14 | |
| edd gene | Eda d | N°15 | See WO2005073364 |
| | Zwf r | N°16 | |
| ldhA gene | ldhAF | N°17 | 1439724 to 1439743 |
| (deletion) | ldhAR | N°18 | 1441029 to 1441007 |
| tpiA gene | YIIQ | N°2 | 4109599 to 4109580 |
| (reconstruction) | tpiA R | N°19 | 4108953 to 4108973 |
| gapA promoter | yeaAF | N°20 | 1860259 to 1860287 |
| (Ptrc16-gapA) | gapAR | N°21 | 1861068 to 1861040 |
| pykA gene | pykAF | N°22 | 1935338 to 1935360 |
| | pykAR | N°23 | 1937425 to 1937401 |
| pykF gene | pykFF | N°24 | 1753371 to 1753392 |
| | pykFR | N°25 | 1755518 to 1755495 |
| gldA gene | YijF D | N°26 | 4135140 to 4135174 |
| | TalCr | N°27 | 4137239 to 4137216 |
| ackA-pta genes | B2295 | N°28 | 2410900 to 2410919 |
| | YfcCR | N°29 | 2415164 to 2415145 |
| poxB gene | poxBF | N°30 | 908475 to 908495 |
| | poxBR | N°31 | 910375 to 910352 |

b) Construction of a Modified Strain E. coli MG1655 lpd*, ΔtpiA, ΔpflAB, ΔadhE, ΔldhA::Cm, ΔgloA, ΔaldA, ΔaldB, Δedd

In order to eliminate the kanamycin resistance cassette and to inactivate the ldhA gene, the chloramphenicol resistance cassette was inserted into the ldhA gene deleting most of the gene concerned according to Protocol 2.

Protocol 2 : Introduction of a PCR Product for Recombination and Selection of the Recombinants

The oligonucleotides chosen and given in Table 2 for replacement of a gene or an intergenic region were used to amplify either the chloramphenicol resistance cassette from the plasmid pKD3 or the kanamycin resistance cassette from the plasmid pKD4 (Datsenko, K. A. & Wanner, B. L. (2000)). The PCR product obtained was then introduced by electroporation into the recipient strain bearing the plasmid pKD46 in which the system λ Red (γ, β, exo) expressed greatly favours homologous recombination. The antibiotic-resistant transformants were then selected and the insertion of the resistance cassette was checked by PCR analysis with the appropriate oligonucleotides given in Table 1.

The other modifications of the strain were checked with the oligonucleotides given in Table 1.

The resulting strain was named E. coli MG1655 lpd*, ΔldhA::Cm, ΔtpiA, ΔpflAB, ΔadhE, ΔgloA, ΔaldA, ΔaldB, Δedd.

At each step, the presence of the modifications previously built in the strain was checked using the oligonucleotides given in Table 1.

TABLE 2
Oligonucleotides used for replacement of a chromosomal region by
recombination with a PCR product in the strain E. coli MG1655

| | | | Homology with |
|---|---|---|---|
| Region name | Names of oligos | SEQ ID | chromosomal region |
| ldhA gene | DldhAF | N°32 | 1440865-1440786 |
| | DldhAR | N°33 | 1439878-1439958 |
| tpiA gene | tpiA::kmF | N°34 | 4109264-4109195 |
| (reconstruction) | tpiA::kmR | N°35 | 4109109-4109193 |
| gapA promoter | Ptrc-gapAF | N°36 | 1860478-1860536 |
| (Ptrc16-gapA) | Ptrc-gapAR | N°37 | 1860762-1860800 |
| pykA gene | DpykAF | N°38 | 1935756-1935836 |
| | DpykAR | N°39 | 1937055-1937135 |
| pykF gene | DpykFF | N°40 | 1753689-1753766 |
| | DpykFR | N°41 | 1755129-1755051 |
| gldA gene | gldA D f | N°42 | 4135511-4135590 |
| | gldA D r | N°43 | 4136615-4136536 |
| ackA-pta genes | DackAF | N°44 | 2411494-2411573 |
| | DptaR | N°45 | 2414906-2414830 |
| poxB gene | DpoxBF | N°46 | 908557-908635 |
| | DpoxBR | N°47 | 910262-910180 |

### Example 2

### Evolution of the Modified Strains E. coli MG1655 lpd* ΔtpiA, ΔpflAB, ΔadhE, ΔldhA::Cm, ΔgloA, ΔaldA, ΔaldB, Δedd in Chemostat Culture Under Anaerobic or Microaerobic Conditions and Physiological Characterization of Evolution

To evolve it toward improved acetol or 1,2 propanediol production, the strain E. coli MG1655 lpd* ΔtpiA, ΔpflAB, ΔadhE, ΔldhA::Cm, ΔgloA, ΔaldA, ΔaldB, Δedd was cultivated in continuous culture under anaerobic conditions on one side and under micro aerobic conditions (1% oxygen) on the other side in the culture medium MPG such as described previously, with excess glucose (from 20 g/l initially with addition if the glucose becomes exhausted). The temperature was set at 37° C. and the pH was regulated at 6.5 by addition of base. The evolution of the strain in the chemostats was followed by the increase of the biomass concentration coupled with the increase of the concentrations of the products, acetol or 1,2-propanediol and the co-product acetate, over several weeks (from 4 weeks up to 6 months). This denoted the improvement of the performances of the strains. When the cultures reached a steady state with no further increase of the concentrations under these conditions, the evolution was done.

The characteristics of the strains before and after evolution were assessed. Single colonies representing individual clones were isolated on Petri dishes. These clones were assessed using the initial strain as control in an Erlenmeyer flask assay, using the same medium MPG used in the chemostat culture. Among these clones, several presented better acetol+1,2-propanediol specific production rates as compared to the control. These clones were selected for the following steps. The results obtained on the best clone for each condition of evolution are reported in Table 4 and 5 below.

TABLE 3
Comparison of the best evolved clone obtained after evolution
under anaerobic conditions with the initial strain

| Strain E. coli MG1655 lpd* ΔtpiA | Initial strain before evolution | Best evolved clone |
|---|---|---|
| ΔpflAB ΔadhE ΔldhA::Cm ΔgloA Δald, | (performances measured after | (performances measured after |
| ΔaldB Δedd | 2 days of culture) | 2 days of culture) |
| Glucose specific consumption rate | 0.12 | 0.21 (+75%) |
| (g glucose/g biomass/h) | | |
| 1,2-propanediol specific production rate | 0.02 | 0.07 (+250%) |
| (g 1,2-propanediol/g biomass/h) | | |
| 1,2-propanediol + hydroxyacetone | 0.04 | 0.08 (+100%) |
| specific production rate | | |
| (g 1,2-propanediol + hydroxyacetone/g | | |
| biomass/h) | | |

TABLE 4
Comparison of the best evolved clone obtained after evolution
under microaerobic conditions with the initial strain

| Strain E. coli MG1655 lpd* ΔtpiA | Initial strain before evolution | Best evolved clone |
|---|---|---|
| ΔpflAB ΔadhE ΔldhA::Cm ΔgloA Δald, | (performances measured after | (performances measured after |
| ΔaldB Δedd | 2 days of culture) | 2 days of culture) |
| Glucose specific consumption rate | 0.10 | 0.22 (+120%) |
| (g glucose/g biomass/h) | | |
| 1,2-propanediol specific production rate | 0.01 | 0.08 (+700%) |
| (g 1,2-propanediol/g biomass/h) | | |
| 1,2-propanediol + hydroxyacetone | 0.04 | 0.08 (+100%) |
| specific production rate | | |
| (g 1,2-propanediol + hydroxyacetone/g | | |
| biomass/h) | | |

As these clones have been cultivated over an extended period of time in culture medium with yeast extract, they needed to be adapted for the growth in minimal medium. The two best clones whose performances are given in Table 3 and 4 were adapted by serial culture on minimal medium in order to increase their growth rates under such conditions and the adaptation was stopped when their growth rates were stable. Clones from the final culture were isolated and checked to be representative of the adapted population.

### Example 3

### Reconstruction of tpiA Gene in the Selected Evolved Strain of E. coli MG1655 lpd*, ΔtpiA, ΔpflAB, ΔadhE, ΔldhA::Cm, ΔgloA, ΔaldA, ΔaldB, Δedd

a) Construction of a Modified Strain E. coli MG1655 tpiA::Km

A kanamycin antibiotic resistance cassette was inserted upstream of the gene tpiA according to the technique described in Protocol 2 with the oligonucleotides given in Table 2. The resulting strain was named E coli MG1655 tpiA::Km.

Then the reconstruction of the gene tpiA into the evolved strain E. coli MG1655 lpd*, ΔtpiA, ΔplfAB, ΔadhE, ΔldhA::Cm, ΔgloA, ΔaldA, ΔaldB, Δedd, ΔarcA, Δndh was performed using the transduction technique with phage P1 described in Protocol 3.

Protocol 3 : Transduction with Phage P1 for Deletion of a Gene

The deletion of the chosen gene by replacement of the gene by a resistance cassette (kanamycin or chloramphenicol) in the recipient E. coli strain was performed by the technique of transduction with phage P1. The protocol was in two steps, (i) the preparation of the phage lysate on the strain MG1655 with a single gene deleted and (ii) the transduction of the recipient strain by this phage lysate.

Preparation of the Phage Lysate
Seeding with 100 μl of an overnight culture of the strain MG1655 with a single gene deleted of 10 ml of LB+Cm 30 μg/ml+glucose 0.2%+CaCl₂ 5 mM.
Incubation for 30 min at 37° C. with shaking.
Addition of 100 μl of phage lysate P1 prepared on the wild type strain MG1655 (approx. 1×10⁹ phage/ml).
Shaking at 37° C. for 3 hours until all cells were lysed.
Addition of 200 ml of chloroform, and vortexing.
Centrifugation for 10 min at 4500 g to eliminate cell debris.
Transfer of supernatant in a sterile tube and addition of 200 μl of chloroform.
Storage of the lysate at 4° C.

Transduction
Centrifugation for 10 min at 1500 g of 5 ml of an overnight culture of the E. coli recipient strain in LB medium.
Suspension of the cell pellet in 2.5 ml of MgSO₄ 10 mM, CaCl₂ 5 mM.
Control tubes: 100 μl cells
   100 μl phages P1 of the strain MG1655 with a single gene deleted.
Tube test: 100 μl of cells+100 μl phages P1 of strain MG1655 with a single gene deleted
Incubation for 30 min at 30° C. without shaking.
Addition of 100 μl sodium citrate 1 M in each tube, and vortexing.
Addition of 1 ml of LB.
Incubation for 1 hour at 37° C. with shaking
Plating on dishes LB+Cm 30 μg/ml after centrifugation of tubes for 3 min at 7000 rpm.
Incubation at 37° C. overnight.

The antibiotic-resistant transformants were then selected and the insertion of the deletion was checked by a PCR analysis with the appropriate oligonucleotides given in Table 1.

The resulting strain was named evolved E. coli MG1655 lpd*, tpiArc::Km, ΔplfAB, ΔadhE, ΔldhA::Cm, ΔgloA, ΔaldA, ΔaldB, Δedd, ΔarcA, Δndh.

The kanamycin and chloramphenicol resistance cassettes were then eliminated according to Protocol 2. The strain obtained was named ‘evolved E. coli tpiArc’

The presence of the modifications previously built in the strain was checked using the oligonucleotides given in Table 1.

### Example 4

### Modifications of the ‘Evolved E. coli tpiArc’: Attenuation of the gapA Gene; Deletion of the Genes pykA and pykF; Deletion of the gldA gene; Over-Expression of ppsA Gene with a Vector pJB137-PgapA-ppsA

a) Replacement of the Natural gapA Promoter with the Synthetic Short Ptrc16 Promoter:

The replacement of the natural gapA promoter with the synthetic short Ptrc16 promoter (SEQ ID NO 48: gagctgttgacgattaatcatccggctcgaataatgtgtgg) into the strain ‘evolved E. coli tpiArc’ was made by replacing 225 pb of upstream gapA sequence with FRT-Cm-FRT and an engineered promoter. The technique used is described in Protocol 2 with the oligonucleotides given in Table 2. The resulting strain was named ‘evolved E. coli tpiArc’ Ptrc16-gapA::Cm.

The chloramphenicol resistance cassette was then eliminated according to Protocol 1. The strain obtained was named ‘evolved E. coli tpiArc’ Ptrc16-gapA.

b) Deletion of the pykA Gene

The gene pykA is inactivated by inserting a kanamycin antibiotic resistance cassette and deleting most of the gene concerned using the technique described in Protocol 2 with the oligonucleotides given in Table 2. The resulting strain is named ‘evolved E. coli tpiArc’ Ptrc16-gapA ΔpykA::Km.

The kanamycin resistance cassette is then eliminated according to Protocol 1. The strain obtained is named ‘evolved E. coli tpiArc’ Ptrc16-gapA ΔpykA.

c) Deletion of the pykF Gene

The gene pykF is inactivated by inserting a kanamycin antibiotic resistance cassette and deleting most of the gene concerned using the technique described in Protocol 2 with the oligonucleotides given in Table 2. The resulting strain is named ‘evolved E. coli tpiArc’ Ptrc16-gapA, ΔpykA ΔpykF::Km.

As previously, the kanamycin resistance cassette is then eliminated according to Protocol 1. The strain obtained is named ‘evolved E. coli tpiArc’ Ptrc16-gapA, ΔpykA, ΔpykF.

d) Deletion of the gldA Gene

Construction of a Modified Strain E. coli MG1655 ΔgldA::Cm

The gene gldA is inactivated by inserting a chloramphenicol antibiotic resistance cassette and deleting most of the genes concerned using the technique described in Protocol 2 with the oligonucleotides given in Table 2. The resulting strain is named E coli MG1655 ΔgldA::Cm.

Construction of a Strain ‘Evolved E. coli tpiArc’ Ptrc16-gapA, ΔpykA, ΔpykF, ΔgldA

The deletion of the gene gldA by replacement of the gene by a chloramphenicol resistance cassette in the strain ‘evolved E. coli tpiArc’ Ptrc16-gapA, ΔpykA, ΔpykF is performed as previously using the transduction technique with phage P1 described in protocol 3. The resulting strain is named ‘evolved E. coli tpiArc’ Ptrc61-gapA, ΔpykA, ΔpykF, ΔgldA::Cm.

The chloramphenicol resistance cassette is then eliminated according to Protocol 1. The strain obtained is named ‘evolved E. coli tpiArc’ Ptrc16-gapA, ΔpykA, ΔpykF, ΔgldA.

e) Introduction of an Expression Vector pJB137-PgapA-ppsA into the Strain

To increase the production of phosphoenolpyruvate the ppsA gene was expressed from the plasmid pJB137 using the gapA promoter. For the construction of plasmid pJB137-PgapA-ppsA, the gene ppsA was PCR amplified from genomic DNA of E. coli MG1655 using the following oligonucleotides:

1. gapA-ppsAF, consisting of 65 bases (SEQ ID NO 49)

ccttttattcactaacaaatagctggtggaatatATGTCCAACAATGGCT
CGTCACCGCTGGTGC

with:
a region (upper-case letters) homologous to the sequence (1785106-1785136) of the gene ppsA (1785136 to 1782758), a reference sequence on the website http://genolist.pasteur.fr/Colibri/), and
a region (lower letters) homologous to the gapA promoteur (1860794-1860761).

2. ppsAR, consisting of 43 bases (SEQ ID NO 50)

aatcgcaagcttGAATCCGGTTATTTCTTCAGTTCAGCCAGGC

with:
a region (upper letters) homologous to the sequence (1782758-1782780) the region of the gene ppsA (1785136 to 1782758)
a restriction site HindIII (underlined letters)
   At the same time the gapA promoter region of the E. coli gene gapA was amplified using the following oligonucleotides:

1. gapA-ppsAR, consisting of 65 bases (SEQ ID NO 51)

GCACCAGCGGTGACGAGCCATTGTTGGACATatattccaccagctatttg
ttagtgaataaaagg

with:
a region (upper-case letters) homologous to the sequence (1785106-1785136) of the gene ppsA (1785136 to 1782758), and
a region (lower letters) homologous to the gapA promoteur (1860794-1860761).

2. gapAF, consisting of 33 bases (SEQ ID NO 52)

ACGTCCCGGGcaagcccaaaggaagagtgaggc

with:
a region (lower letters) homologous to the gapA promoteur (1860639-1860661).
a restriction site SmaI (underlined letters)
   Both fragments were subsequently fused using the oligonucleotides ppsAR and gapAF (Horton et al. 1989 Gene 77:61-68). The PCR amplified fragment were cut with the restriction enzymes HindIII and SmaI and cloned into the HindIII/SmaI sites of the vector pJB137 (EMBL Accession number: U75326) giving vector pJB137-PgapA-ppsA. Recombinant plasmids were verified by DNA sequencing.

The plasmid pJB137-PgapA-ppsA is introduced into the strain ‘evolved E. coli tpiArc’ Ptrc16-gapA, ΔpykA, ΔpykF, ΔgldA.

The strain obtained is named ‘evolved E. coli tpiArc’, Ptrc16-gapA, ΔpykA, ΔpykF, ΔgldA (pJB137-PgapA-ppsA).

At each step, the presence of the modifications previously built in the strain was checked using the oligonucleotides given in Table 1.

### Example 5

### Construction of a Strain ‘Evolved E. coli tpiArc’ Ptrc16-gapA, ΔpykA, ΔpykF, ΔgldA, ΔackA-pta, ΔpoxB (pJB137-PgapA-ppsA) Able to Produce Acetol Without Acetate as By-Product

a) Construction of a Modified Strain E. coli MG1655 ΔackA-pta::Cm

The genes ackA and pta are inactivated by inserting a chloramphenicol antibiotic resistance cassette and deleting most of the gene concerned using the technique described in Protocol 2 with the oligonucleotides given in Table 2. The resulting strain is named E coli MG1655 ΔackA-pta::Cm.

b) Construction of a Strain ‘Evolved E. coli tpiArc’ Ptrc16-gapA, ΔpykA, ΔpykF, ΔgldA, ΔackA-pta

The deletion of the genes ackA and pta in the strain ‘evolved E. coli tpiArc’ Ptrcl-gapA, ΔpykA, ΔpykF is performed as previously using the transduction technique with phage P1 as described in Protocol 3.

The resulting strain is named ‘evolved E. coli tpiArc’ Ptrc16-gapA, ΔpykA, ΔpykF, ΔgldA, ΔackA-pta::Cm.

As previously, the chloramphenicol resistance cassette is then eliminated according to Protocol 1. The strain obtained is named ‘evolved E. coli tpiArc’ Ptrc16-gapA, ΔpykA, ΔpykF, ΔgldA, ΔackA-pta.

c) Construction of a Modified Strain ‘evolved E. coli tpiArc’ Ptrcl-gapA, ΔpykA, ΔpykF, ΔgldA , ΔackA-pta, ΔpoxB (pJB137-PgapA-ppsA)

The gene poxB is inactivated by inserting a chloramphenicol antibiotic resistance cassette and deleting most of the gene concerned using the technique described in Protocol 2 with the oligonucleotides given in Table 2.

The resulting strain is named evolved E. coli tpiArc Ptrc16-gapA, ΔpykA, ΔpykF, ΔgldA, ΔackA-pta, ΔpoxB::Cm.

As previously, the chloramphenicol resistance cassette is then eliminated according to protocol 1. The strain obtained is named evolved E. coli tpiArc Ptrc16-gapA, ΔpykA, ΔpykF, ΔgldA, ΔackA-pta, ΔpoxB.

The plasmid pJB137-PgapA-ppsA is introduced into the strain evolved E. coli tpiArc Ptrcl-gapA, ΔpykA, ΔpykF, ΔgldA, ΔackA-pta, ΔpoxB. The strain obtained is named evolved E. coli tpiArc Ptrc16-gapA, ΔpykA, ΔpykF, ΔgldA , ΔackA-pta, ΔpoxB (pJB137-PgapA-ppsA).

At each step, the presence of the modifications previously built in the strain is checked using the oligonucleotides given in Table 1.

### Example 6

### Comparison of the Different Evolved Strains for Acetol Production Under Aerobic Conditions

The strains obtained as described in Example 4 and the control strains (control 1: MG1655 lpd* ΔtpiA ΔpflAB ΔadhE ΔldhA::Cm ΔgloA Δald, ΔaldB Δedd evolved under anaerobic conditions and control 2: MG1655 lpd* ΔtpiA ΔpflAB ΔadhE ΔldhA::Cm ΔgloA Δald, ΔaldB Δedd evolved under microaerobic conditions) were cultivated in an Erlenmeyer flask assay under aerobic conditions in minimal medium supplemented with yeast extract and with glucose as carbon source. The culture was carried out at 34° C. and the pH was maintained by buffering the culture medium with MOPS. At the end of the culture, acetol, 1,2-propanediol and residual glucose in the fermentation broth were analysed by HPLC and the yields of acetol over glucose were calculated. The best strain is then selected for a fermenter fed-batch culture.

| | Acetol titer | Acetol yield |
|---|---|---|
| Strain | (g/l) | (g/g glucose) |
| Control 1 | 3.25 | 0.34 |
| Control 2 | 2.84 | 0.30 |
| ‘evolved E. coli tpiArc’, Ptrc16-gapA, | 3.19 | 0.40 |
| (pJB137-PgapA-ppsA) | | |
| (built from control 1) | | |
| ‘evolved E. coli tpiArc’, Ptrc16-gapA, | 3.65 | 0.38 |
| (pJB137-PgapA-ppsA) | | |
| (built from control 2) | | |

1,2-propanediol titers in the cultures were below 0.4 g/l.

### Example 7

### Production of Acetol in Fed-Batch Culture with the Best Strain

The best strain selected in the previous experiment is cultivated in a 21 fermenter using a fed-batch protocol.

The temperature of the culture is maintained constant at 37° C. and the pH is permanently adjusted to values between 6.5 and 8 using an NH₄OH solution. The agitation rate is maintained between 200 and 300 rpm during the batch phase and is increased to up to 1000 rpm at the end of the fed-batch phase. The concentration of dissolved oxygen is maintained at values between 30 and 40% saturation by using a gas controller. When the optical density reachs a value between three and five, the fed-batch is started with an initial flow rate between 0.3 and 0.5 ml/h, and a progressive increase up to flow rate values between 2.5 and 3.5 ml/h. At this point the flow rate is maintained constant for 24 to 48 hours. The medium of the fed is based on minimal media containing glucose at concentrations between 300 and 500 g/l.

1. A method for the preparation of an evolved strain of microorganism for the production of acetol from a simple carbon source, said method comprising:
growing an initial strain under selection pressure in an appropriate growth medium, said initial bacterial strain comprising an attenuation of the expression of the tpiA gene and an attenuation of the expression of at least one gene involved in the conversion of methylglyoxal into lactate, in order to promote evolution in said initial strain,
then selecting and isolating the evolved strain having an increased acetol or 1,2 propanediol production rate,
then reconstructing a functional tpiA gene in the evolved strain.

2. The method of claim 1, wherein the gene involved in conversion of methylglyoxal into lactate is selected from the group consisting of: gloA, aldA, aldB and combinations thereof.

3. The method of claim 1, wherein the initial strain comprises furthermore the attenuation of expression of at least one of the genes selected from the group consisting of ldhA, pflA, pflB, adhE, edd and eda.

4. The method of claim 1 wherein the evolved strain is selected and isolated on the basis of an acetol and/or 1,2-propanediol production rate, increased by at least 20% compared to the production rate of the initial strain.

5. The method of claim 1, wherein the initial strain is selected from the group consisting of bacteria, yeasts and fungi.

6. The method of claim 5, wherein the initial strain is selected from the group consisting of Enterobacteriaceae, Bacillaceae, Streptomycetaceae and Corynebacteriaceae.

7. The method of claim 6, wherein the initial strain is either Escherichia coli or Klebsiella pneumoniae.

8. An evolved strain of microorganism capable of being obtained by the method according to claim 1.

9. The evolved strain according to claim 8 wherein glyceraldehyde 3 phosphate dehydrogenase activity is attenuated.

10. The evolved strain according to claim 9 wherein expression of the gapA gene is attenuated.

11. The evolved strain according to claim 8 wherein efficiency of the sugar import is increased.

12. The evolved strain according to claim 11 wherein a sugar import system independent of phosphoenolpyruvate is used.

13. The evolved strain according to claim 12 wherein expression of at least one gene selected among galP and glk is increased.

14. The evolved strain according to claim 11 wherein efficiency of the sugar-phosphotransferase system is improved by increasing availability of the metabolite phosphoenolpyruvate.

15. The evolved strain of claim 14 wherein expression of at least one pyruvate kinase is attenuated.

16. The evolved strain according to claim 15 wherein the expression of at least one gene selected among pykA and pykF is attenuated.

17. The evolved strain according to claim 14 wherein phosphoenolpyruvate synthase activity is increased.

18. The evolved strain according to claim 17 wherein expression of the ppsA gene is increased.

19. The evolved strain according to claim 1 wherein activity of at least one enzyme involved in the conversion of acetol into 1,2-propanediol is attenuated.

20. The evolved strain of claim 19 wherein expression of the gldA gene is attenuated.

21. The evolved strain according to claim 8 wherein at least one enzyme involved in synthesis of acetate is attenuated.

22. The evolved strain according to claim 21 wherein expression of at least one gene selected among ackA, pta, poxB is attenuated.

23. A method for preparing acetol wherein an evolved strain according to claim 8 is grown in an appropriate growth medium comprising a simple carbon source, and produced acetol is recovered.

24. The method of claim 23, wherein the recovered acetol is furthermore purified.

25. A method for the preparation of an evolved strain of microorganism suitable for production of acetol from a simple carbon source, said method comprising:
growing an initial strain under selection pressure in an appropriate growth medium, said initial bacterial strain comprising
an attenuation of the expression of the tpiA gene, and
an attenuation of at least one of the genes selected from the group consisting of: gloA, aldA and aldB, and
an attenuation of the expression of at least one of the genes selected from the group consisting of ldhA, pflA, pflB, adhE, edd and eda, in order to promote evolution in said initial strain,
then selecting and isolating the evolved strain having an increased acetol or 1,2 propanediol production rate,
then reconstructing a functional tpiA gene in the evolved strain.

26. The method of claim 25 wherein the initial strain comprises at least one of the following:
the tpiA gene is deleted, and
the genes gloA, aldA and aldB are deleted, and
the genes ldhA, pflA, adhE and edd are deleted.

27. An evolved strain of microorganism obtainable by the method according to claim 25.

28. An evolved strain of microorganism obtainable by the method according to claim 26.

29. A method for preparing acetol wherein an evolved strain according to claim 27 is grown in an appropriate growth medium obtainable a simple carbon source, and produced acetol is recovered.

30. A method for preparing acetol wherein an evolved strain according to claim 28 is grown in an appropriate growth medium obtainable a simple carbon source, and produced acetol is recovered.

31. The method of claim 29, wherein the recovered acetol is furthermore purified.


SEQUENCE LISTING (52 sequences)

SEQ ID NO: 1 - DNA, 20 bp, Artificial Sequence
  Oligonucleotide
ggtgatgatagttatcgccg

SEQ ID NO: 2 - DNA, 20 bp, Artificial Sequence
  Oligonucleotide
cgtgccatcgacagcagtcc

SEQ ID NO: 3 - DNA, 30 bp, Artificial Sequence
  Oligonucleotide
agacattaaaaatatacgtgcagctacccg

SEQ ID NO: 4 - DNA, 30 bp, Artificial Sequence
  Oligonucleotide
gtgaaagctgacaacccttttgatctttta

SEQ ID NO: 5 - DNA, 22 bp, Artificial Sequence
  Oligonucleotide
ggctcattgcaccaccatccag

SEQ ID NO: 6 - DNA, 24 bp, Artificial Sequence
  Oligonucleotide
gaaaagacgcgctgacaatacgcc

SEQ ID NO: 7 - DNA, 20 bp, Artificial Sequence
  Oligonucleotide
gccatcagcaggcttagccg

SEQ ID NO: 8 - DNA, 23 bp, Artificial Sequence
  Oligonucleotide
gggtattgtggcatgtttaaccg

SEQ ID NO: 9 - DNA, 31 bp, Artificial Sequence
  Oligonucleotide
gaagtggtcgatgccgggattgaagaatggg

SEQ ID NO: 10 - DNA, 31 bp, Artificial Sequence
  Oligonucleotide
gggttacgtttcagtgaggcgcgttctgcgg

SEQ ID NO: 11 - DNA, 31 bp, Artificial Sequence
  Oligonucleotide
tgcagcggcgcacgatggcgacgttccgccg

SEQ ID NO: 12 - DNA, 31 bp, Artificial Sequence
  Oligonucleotide
cacgatgacgaccattcatgcctatactggc

SEQ ID NO: 13 - DNA, 40 bp, Artificial Sequence
  Oligonucleotide
catatttccctcaaagaatataaaaaagaacaattaacgc

SEQ ID NO: 14 - DNA, 31 bp, Artificial Sequence
  Oligonucleotide
tatgttcatgcgatggcgcaccagctgggcg

SEQ ID NO: 15 - DNA, 24 bp, Artificial Sequence
  Oligonucleotide
ccccggaatcagaggaatagtccc

SEQ ID NO: 16 - DNA, 29 bp, Artificial Sequence
  Oligonucleotide
gggtagactccattactgaggcgtgggcg

SEQ ID NO: 17 - DNA, 20 bp, Artificial Sequence
  Oligonucleotide
gccatcagcaggcttagcgc

SEQ ID NO: 18 - DNA, 23 bp, Artificial Sequence
  Oligonucleotide
gggtattgtggcatgtttaaccg

SEQ ID NO: 19 - DNA, 21 bp, Artificial Sequence
  Oligonucleotide
catttccggtggtgcgattgc

SEQ ID NO: 20 - DNA, 29 bp, Artificial Sequence
  Oligonucleotide
gccacagccggaatcatacttggtttggg

SEQ ID NO: 21 - DNA, 29 bp, Artificial Sequence
  Oligonucleotide
cgtcaacaccaacttcgtcccatttcagg

SEQ ID NO: 22 - DNA, 23 bp, Artificial Sequence
  Oligonucleotide
ggcaattaccctcgacgtaccgg

SEQ ID NO: 23 - DNA, 25 bp, Artificial Sequence
  Oligonucleotide
ccgatggatgatctgttagaggcgg

SEQ ID NO: 24 - DNA, 22 bp, Artificial Sequence
  Oligonucleotide
gcgtaaccttttccctggaacg

SEQ ID NO: 25 - DNA, 24 bp, Artificial Sequence
  Oligonucleotide
gcgttgctggagcaacctgccagc

SEQ ID NO: 26 - DNA, 35 bp, Artificial Sequence
  oligonucleotide
gcctggatttgtaccacggttggtggaacggcggg

SEQ ID NO: 27 - DNA, 24 bp, Artificial Sequence
  oligonucleotide
cacgcatattccccattgccgggg

SEQ ID NO: 28 - DNA, 20 bp, Artificial Sequence
  Oligonucleotide
gcatgggtaaacttaaggcg

SEQ ID NO: 29 - DNA, 20 bp, Artificial Sequence
  Oligonucleotide
taatcaccaacgtatcgggc

SEQ ID NO: 30 - DNA, 21 bp, Artificial Sequence
  Oligonucleotide
cgcggcttggtcgggtaacgg

SEQ ID NO: 31 - DNA, 24 bp, Artificial Sequence
  Oligonucleotide
tcgggctatttaaccgttagtgcc

SEQ ID NO: 32 - DNA, 100 bp, Artificial Sequence
  Oligonucleotide
gaaactcgccgtttatagcacaaaacagtacgacaagaagtacctgcaacaggtgaacgagtcctttggctttgagctggtgtaggctggagctgcttcg

SEQ ID NO: 33 - DNA, 101 bp, Artificial Sequence
  Oligonucleotide
ttaaaccagttcgttcgggcaggtttcgcctttttccagattgcttaagttttgcagcgtagtctgagaaatactggtcagcatatgaatatcctccttag

SEQ ID NO: 34 - DNA, 124 bp, Artificial Sequence
  Oligonucleotide
ggggctgaccttcgctgttgaaccgattaagctggcgctatctgaatcgcttgaaggtttgaataaatgatcacactggctcaccttcgggtgggcctttctgccatatgaatatcctccttag

SEQ ID NO: 35 - DNA, 106 bp, Artificial Sequence
  Oligonucleotide
gcgaataaaggaagatggccgccccgcagggcagcaggtctgtgaaacagtatagagattcatcggcacaaaggctttgctttttgtgtaggctggagctgcttcg

SEQ ID NO: 36 - DNA, 100 bp, Artificial Sequence
  Oligonucleotide
agtcatatattccaccagctatttgttagtgaataaaagccacacattattcgagccggatgattaatagtcaacagctctgtaggctggagctgcttcg

SEQ ID NO: 37 - DNA, 79 bp, Artificial Sequence
  Oligonucleotide
gctcacattacgtgactgattctaacaaaacattaacaccaactggcaaaattttgtcccatatgaatatcctccttag

SEQ ID NO: 38 - DNA, 101 bp, Artificial Sequence
  Oligonucleotide
cgcggcgggtgccaacgttgtacgtatgaacttttctcacggctcgcctgaagatcacaaaatgcgcgcggataaagttcgtgtaggctggagctgcttcg

SEQ ID NO: 39 - DNA, 101 bp, Artificial Sequence
  Oligonucleotide
cgccgcatccggcaacgtacttactctaccgttaaaatacgcgtggtattagtagaacccacggtactcatcacgtcgccccatatgaatatcctccttag

SEQ ID NO: 40 - DNA, 98 bp, Artificial Sequence
  Oligonucleotide
cccatccttctcaacttaaagactaagactgtcatgaaaaagaccaaaattgtttgcaccatcggaccgaaaaccgaatgtaggctggagctgcttcg

SEQ ID NO: 41 - DNA, 99 bp, Artificial Sequence
  Oligonucleotide
ggacgtgaacagatgcggtgttagtagtgccgctcggtaccagtgcaccagaaaccataactacaacgtcacctttgtgcatatgaatatcctccttag

SEQ ID NO: 42 - DNA, 101 bp, Artificial Sequence
  oligonucleotide
gttattcccactcttgcaggaaacgctgaccgtactggtcggctaccagcagagcggcgtaaacctgatctggcgtcgcggtgtaggctggagctgcttcg

SEQ ID NO: 43 - DNA, 100 bp, Artificial Sequence
  oligonucleotide
atggaccgcattattcaatcaccgggtaaatacatccagggcgctgatgtgattaatcgtctgggcgaatacctgaagcccatatgaatatcctccttag

SEQ ID NO: 44 - DNA, 100 bp, Artificial Sequence
  Oligonucleotide
cgagtaagttagtactggttctgaactgcggtagttcttcactgaaatttgccatcatcgatgcagtaaatggtgaagagtgtaggctggagctgcttcg

SEQ ID NO: 45 - DNA, 97 bp, Artificial Sequence
  Oligonucleotide
gctgctgtgcagactgaatcgcagtcagcgcgatggtgtagacgatatcgtcaaccagtgcgccacgggacaggtcgcatatgaatatcctccttag

SEQ ID NO: 46 - DNA, 99 bp, Artificial Sequence
  Oligonucleotide
ccttagccagtttgttttcgccagttcgatcacttcatcaccgcgtccgctgatgattgcgcgcagcatatacaggctgcatatgaatatcctccttag

SEQ ID NO: 47 - DNA, 102 bp, Artificial Sequence
  Oligonucleotide
cggttgcagcttatatcgccaaaacactcgaatcggcaggggtgaaacgcatctggggagtcacaggcgactctctgaacggtgtaggctggagctgcttcg

SEQ ID NO: 48 - DNA, 41 bp, Artificial Sequence
  Synthetic promoter
gagctgttgacgattaatcatccggctcgaataatgtgtgg

SEQ ID NO: 49 - DNA, 65 bp, Artificial Sequence
  Oligonucleotide
ccttttattcactaacaaatagctggtggaatatatgtccaacaatggctcgtcaccgctggtgc

SEQ ID NO: 50 - DNA, 43 bp, Artificial Sequence
  Oligonucleotide
aatcgcaagcttgaatccggttatttcttcagttcagccaggc

SEQ ID NO: 51 - DNA, 65 bp, Artificial Sequence
  Oligonucleotide
gcaccagcggtgacgagccattgttggacatatattccaccagctatttgttagtgaataaaagg

SEQ ID NO: 52 - DNA, 33 bp, Artificial Sequence
  Oligonucleotide
acgtcccgggcaagcccaaaggaagagtgaggc